(19) United States
(12) Patent Application Publication
(10) Pub. No.: US 2026/0057510 A1
(43) Pub. Date: Feb. 26, 2026

(54) METHODS AND RELATED ASPECTS FOR PRODUCING PROCESSED IMAGES WITH CONTROLLED IMAGES QUALITY LEVELS

(71) Applicant: THE JOHNS HOPKINS UNIVERSITY, Baltimore, MD (US)

(72) Inventors: Joseph Webster STAYMAN, Baltimore, MD (US); Jianan GANG, Baltimore, MD (US); Jeremias SULAM, Baltimore, MD (US); Wenying WANG, Baltimore, MD (US)

(21) Appl. No.: 19/102,025

(22) PCT Filed: Aug. 8, 2023
(86) PCT No.: PCT/US2023/029692

§ 371 Date: Feb. 7, 2025

Related U.S. Application Data
(60) Provisional application No. 63/396,685, filed on Aug. 10, 2022.
(60) Provisional application No. 63/384,573, filed on Nov. 21, 2022.

(51) Int. Cl. G06T 7/00; A61B 6/00; A61B 6/03; A61B 6/42; A61B 6/58; G06T 5/70; G06T 5/73

(52) U.S. Cl. CPC G06T 7/0012; A61B 6/032; A61B 6/4208; A61B 6/5282; A61B 6/582; G06T 5/70; G06T 5/73; G06T 2207/10081; G06T 2207/20084; G06T 2207/30168; G06T 2207/30196

(57) ABSTRACT

Provided herein are methods of producing processed images having user-controlled image quality levels. In some embodiments, the methods include receiving a selected input value of a control parameter from a user in a trained electronic neural network in which the selected input value determines an image quality level of the processed test image. The image quality level typically comprises relative amounts of a noise measure and a bias measure in the processed test image. The methods also generally include passing test image data through the trained electronic neural network, and outputting from the trained electronic neural network the processed test image. Related systems and computer program products are also provided.

### CROSS-REFERENCE TO RELATED APPLICATIONS

This application is the national stage entry of International Patent Application No. PCT/US2023/029692, filed on Aug. 8, 2023, and published as WO 2024/035674 A1 on Feb. 15, 2024, which claims the benefit of U.S. Provisional Patent Application Ser. Nos. 63/384,573, filed on Nov. 21, 2022 and 63/396,685, filed on Aug. 10, 2022, which are hereby incorporated by reference in their entireties.

### STATEMENT REGARDING FEDERALLY SPONSORED RESEARCH OR DEVELOPMENT

This invention was made with government support under grants EB027127 and CA249538 awarded by the National Institutes of Health. The government has certain rights in the invention.

### BACKGROUND

X-ray computed tomography (CT) has wide use in disease screening, diagnosis, and interventional guidance. Increasing CT use has raised concerns of excessive radiation dose to population and encouraged the development of low-dose techniques. Due to the reduced number of photons in low-dose CT, reconstructed images contain higher noise making detection of small and low-contrast lesions more difficult. Algorithmic techniques including projection denoising, statistical iterative reconstruction, and image denoising have been developed for image quality improvement. Image denoising is applied to reconstructed images and can be easily integrated into existing CT pipelines. Image filters such as nonlocal means and block-matching 3D can reduce noise to a great extent. However, the filters generally do not attempt to directly model the noise distribution and may over-smooth the imaging resulting in loss of structural features. Recently, deep learning techniques have become very popular in image processing including denoising applications. Chen et al. designed a residual encoder-decoder convolutional network (RED-CNN) that suppresses noise and attempts to maintain structural features (Chen et al., “Low-Dose CT With a Residual Encoder-Decoder Convolutional Neural Network,” IEEE Transactions on Medical Imaging 36, 2524-2535 (2017)).

Machine learning approaches differ from traditional processing in many ways. The ability of networks to learn distributions of both the noise properties and the underlying prior distribution of image content has distinct advantages over other approaches. Such methods are often able to provide results that are dramatic improvements over traditional noise-resolution trade-offs in classic methods. However, machine learning methods also differ in that they tend to provide only a single level of performance. That is, there generally is no provision for parametric control of how aggressive the noise reduction should be. While a single image output is convenient, this restricts radiologist preference and the ability to tune the level of noise reduction to the particular diagnostic task. For example, recent work has employed task-based metrics to optimize classic reconstruction methods as well as imaging system design for specific tasks.

Image properties in machine learning approaches can be complex, and difficult to describe in terms of a classic noise-resolution trade-off. However, there is a more general noise-bias trade-off that is being made based on the particular loss function used to train the network. Biases can manifest as blur but also as other misrepresentations of the image volume including the elimination of specific structures or textures, and the injection of false features.

Accordingly, there is a need for additional methods, and related aspects, for producing processed images that have controlled image quality levels.

### SUMMARY

The present disclosure relates, in certain aspects, to methods of producing processed test images having controlled image quality levels. In some aspects, the present disclosure also provides methods of generating electronic neural networks of use in producing processed test images having controlled image quality levels. In some implementations, for example, the present disclosure provides a convolutional neural network (CNN) for low-dose CT denoising with a hyper-parameter a that can be used to control the trade-off between noise reduction and bias. Examples are provided herein where this a induces different spatial resolutions in the denoised image. Optionally, other more general parameterizations of bias can be used. In addition, the sigma-CNN (sCNN) disclosed herein provides a flexible selection of spatial resolution, allowing a user-defined balance between noise and bias for specific tasks. Related systems and computer readable media are also provided.

In one aspect, for example, the present disclosure provides a computer-implemented method of producing a processed test image having a controlled image quality level. The method includes receiving at least one selected input value of at least one control parameter in a trained electronic neural network in which the selected input value determines an image quality level of the processed test image. The image quality level comprises relative amounts of at least one noise measure and at least one bias measure in the processed test image. The method also includes passing test image data through the trained electronic neural network, and outputting from the trained electronic neural network the processed test image, thereby producing the processed test image having the controlled image quality level.

In another aspect, the present disclosure provides a system for producing a processed test image having a controlled image quality level using an electronic neural network. The system includes a processor, and a memory communicatively coupled to the processor, the memory storing instructions which, when executed on the processor, perform operations comprising: receiving at least one selected input value of at least one control parameter in a trained electronic neural network, wherein the selected input value determines an image quality level of the processed test image, which image quality level comprises relative amounts of at least one noise measure and at least one bias measure in the processed test image; passing test image data through the trained electronic neural network; and outputting from the trained electronic neural network the processed test image to thereby produce the processed test image having the controlled image quality level.

In another aspect, the present disclosure provides a computer readable media comprising non-transitory computer executable instruction which, when executed by at least electronic processor, perform at least: receiving at least one selected input value of at least one control parameter in a trained electronic neural network, wherein the selected input value determines an image quality level of the processed test image, which image quality level comprises relative amounts of at least one noise measure and at least one bias measure in the processed test image; passing test image data through the trained electronic neural network; and outputting from the trained electronic neural network the processed test image to thereby produce the processed test image having the controlled image quality level.

Various optional features of the above embodiments include the following. The control parameter represents a level of bias in the processed test image. The control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof. The bias measure comprises a spatial resolution level, a blurriness level, or another misrepresentation of an image volume. The processed test image comprises a denoised image. The trained electronic neural network comprises a loss function having the formula:

$$( μ , \hat{μ} , σ ) = {\vert f ( \hat{μ} , σ ) - G ( σ ) * μ \vert}^{2}$$

where μ is a ground truth image, \hat{μ} is an image estimate using filter-back projection (FBP), σ is the control parameter, and G(σ) is a Gaussian kernel with standard deviation σ. The loss function evaluates a mean squared error (MSE) between the image estimate and the parameterized ground truth image. The trained electronic neural network comprises a convolutional neural network (CNN). The test image data comprises one or more images selected from the group consisting of: a magnetic resonance (MR) image, a computed tomography (CT) image, a single photon emission computed tomography (SPECT) image, a positron emission tomography (PET) image, and a microscopy image. The test image data comprises acquired computed tomography (CT) projection data of an object. The processed test image comprises a reconstructed CT image. The bias measure is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect. The methods include receiving x-ray CT data at one or more x-ray detectors configured and positioned to detect x-ray energy transmitted through the object from one or more x-ray energy sources to generate the acquired CT projection data of the object. The object comprises a subject (e.g., a human subject).

### BRIEF DESCRIPTION OF THE DRAWINGS

The accompanying drawings, which are incorporated in and constitute a part of this specification, illustrate certain embodiments, and together with the written description, serve to explain certain principles of the methods, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 is a flow chart that schematically depicts exemplary method steps of producing a processed test image having a controlled image quality level according to some aspects disclosed herein.

FIG. 2 is a schematic diagram of an exemplary system suitable for use with certain aspects disclosed herein.

FIG. 3 is a schematically shows the architecture of the denoising CNN with spatial resolution control, where the resolution control components are color-coded with orange according to some aspects disclosed herein. The network receives two inputs: 1) the low-dose CT image and 2) a hyper-parameter σ for resolution control. The output is a denoised image at desired spatial resolution.

FIGS. 4A-4C show inserted task objects: (a) a at Gaussian signal (standard deviation=4 pixels), (b) a sharp Gaussian signal (standard deviation=1 pixels). (c) Examples of normal dose CT images with task object insertions. a plot showing an illustration of projection-dependent tube warm up scaling factors applied to the projection data after mean-I₀-correction.

FIG. 5 is one example in the testing set. The normal dose image (ground truth), the simulated low-dose image, and the denoised images with the RED-CNN model or the proposed sCNN model at different resolution levels are displayed. All display windows are [0,0.03]m⁻¹.

FIG. 6 are MSE plots with error bars.

FIG. 7 shows a plot of relative detectability indices of two imaging tasks with respective to a at locations 1 (left) and 2 (right).

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, systems, and component parts, the following terminology, and grammatical variants thereof, will be used in accordance with the definitions set forth below.

Classifier. As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., medical images or the like).

Labeled: As used herein, “labeled” in the context of data sets or points refers to data that is classified as, or otherwise associated with, having or lacking a given characteristic or property.

Machine Learning Algorithm: As used herein, “machine learning algorithm,” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART —classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.”

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

Substantially: As used herein, “substantially,” “about,” or “approximately” as applied to one or more values or elements of interest, refers to a value or element that is similar to a stated reference value or element. In certain embodiments, the term “substantially,” “about,” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Value: As used herein, “value” generally refers to an entry in a dataset that can be anything that characterizes the feature to which the value refers. This includes, without limitation, numbers, words or phrases, symbols (e.g., + or −) or degrees.

### DETAILED DESCRIPTION

A wide range of dose reduction strategies for x-ray computed tomography (CT) have been investigated. Recently, denoising strategies based on machine learning have been widely applied, often with impressive results, and breaking free from traditional noise-resolution trade-offs. However, since typical machine learning strategies provide a single denoised image volume, there is no user-tunable control of a particular trade-off between noise reduction and image properties (biases) of the denoised image. This is in contrast to traditional filtering and model-based processing that permits tuning of parameters for a level of noise control appropriate for the specific diagnostic task. Accordingly, in some aspects, the present disclosure provides electronic neural networks that include control parameters (e.g., spatial-resolution parameters, etc.) as additional input permits explicit control of the noise-bias trade-off. As described herein, these methods and other attributes provide the ability to control image properties through such parameterization as well as the ability to tune such parameters for increased detectability in task-based evaluation, among other attributes. These and other aspects will be apparent upon a complete review of the present disclosure, including the accompanying figures.

### Exemplary Methods

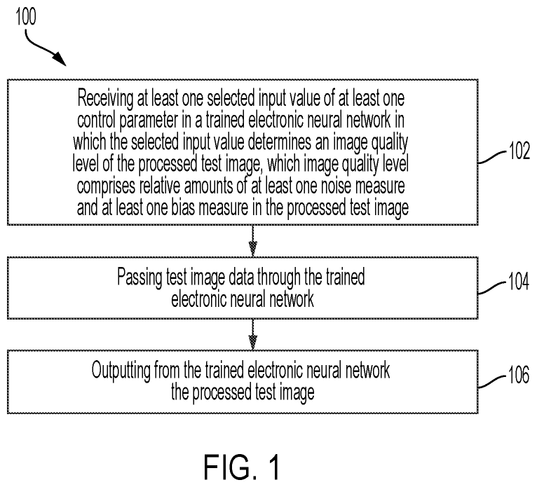

The present disclosure provides various computer-implemented methods of producing processed test images having controlled image quality levels. To illustrate, FIG. 1 is flow chart that schematically depicts exemplary method steps according to some aspects disclosed herein. As shown, method 100 includes receiving at least one selected input value of at least one control parameter in a trained electronic neural network in which the selected input value determines an image quality level of the processed test image (step 102). The image quality level comprises relative amounts of at least one noise measure and at least one bias measure in the processed test image. Method 100 also includes passing test image data through the trained electronic neural network (step 104), and outputting from the trained electronic neural network the processed test image to thereby produce the processed test image having the controlled image quality level (step 106).

The methods and other aspects of the present disclosure include various optional features. In some embodiments, for example, the control parameter represents a level of bias in the processed test image. In some embodiments, the control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof. In some embodiments, the bias measure comprises a spatial resolution level, a blurriness level, or another misrepresentation of an image volume.

In some embodiments, the processed test image comprises a denoised image. In some embodiments, the trained electronic neural network comprises a loss function having the formula:

$$( μ , \hat{μ} , σ ) = {\vert f ( \hat{μ} , σ ) - G ( σ ) * μ \vert}^{2}$$

where μ is a ground truth image, \hat{μ} is an image estimate using filter-back projection (FBP), σ is the control parameter, and G(σ) is a Gaussian kernel with standard deviation σ, In some of these embodiments, the loss function evaluates a mean squared error (MSE) between the image estimate and the parameterized ground truth image. The trained electronic neural network comprises a convolutional neural network (CNN).

In some embodiments, the test image data comprises one or more images selected from the group consisting of: a magnetic resonance (MR) image, a computed tomography (CT) image, a single photon emission computed tomography (SPECT) image, a positron emission tomography (PET) image, and a microscopy image. In some embodiments, for example, the test image data comprises acquired computed tomography (CT) projection data of an object. In some embodiments, the object comprises a subject (e.g., a human subject, etc.). In some embodiments, the processed test image comprises a reconstructed CT image. The bias measure is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect. In some embodiments, the methods include receiving x-ray CT data at one or more x-ray detectors configured and positioned to detect x-ray energy transmitted through the object from one or more x-ray energy sources to generate the acquired CT projection data of the object.

### EXEMPLARY SYSTEMS AND COMPUTER READABLE MEDIA

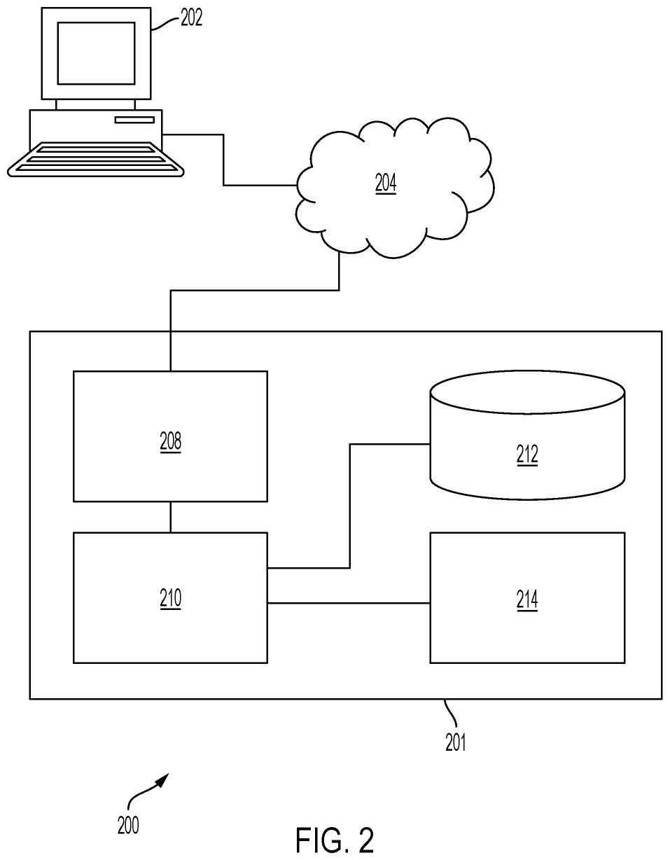

The present disclosure also provides various systems and computer program products or machine readable media. To illustrate, FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic image data records system, such as an LIS, a database, a compendium of clinical data, or any other source of images suitable for use as a training corpus as disclosed herein. As used herein, the image data embraces any type of specimen in any field, not limited to pathology, radiology, or the like where the problem at hand involves labels for groups of components, e.g., a set of radiology images, such as CT image volumes, etc. Due to hardware volatile memory storage limitations, each constituent image may be broken down into a number of tiles, which may be, e.g., 128 pixels by 128 pixels. Such tiles are examples of “components” as that term is used herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 308 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others.

To further illustrate, in certain aspects, this disclosure provides systems that include one or more processors, and one or more memory components in communication with the processor. The memory component typically includes one or more instructions that, when executed, cause the processor to provide information that causes at least one reconstructed CT image and/or the like to be displayed (e.g., via computer 201 or the like).

In some aspects, program products includes non-transitory computer-executable instructions which, when executed by an electronic processor perform at least: receiving at least one selected input value of at least one control parameter in a trained electronic neural network, wherein the selected input value determines an image quality level of the processed test image, which image quality level comprises relative amounts of at least one noise measure and at least one bias measure in the processed test image; passing test image data through the trained electronic neural network; and outputting from the trained electronic neural network the processed test image to thereby produce the processed test image having the controlled image quality level.

System 200 also typically includes additional system components (e.g., CT imaging device) that are configured to perform various aspects of the methods described herein. In some of these aspects, one or more of these additional system components are positioned remote from and in communication with a remote server through an electronic communication network, whereas in other aspects, one or more of these additional system components are positioned local, and in communication with a server (i.e., in the absence of an electronic communication network) or directly with, for example, a desktop computer. Although not within view, in some embodiments, a CT imaging device includes at least one x-ray energy source and at least one x-ray detector configured and positioned to detect x-ray energy transmitted through an object (e.g., a subject or the like) from the x-ray energy source.

### Example: A CT Denoising Neural Network with Image Properties Parameterization and Control

### 1. Methods

1.1. Generalized Formulation for Noise Reduction with Parametric Control

In this example, we adopt a conventional monoenergetic CT model where the measurements y∈ [symbol] are distributed y˜Poisson (I₀exp {−Aμ}), where μ∈ [symbol] is the ground truth image volume, A∈ [symbol] is the projection matrix, and I₀ denotes the nominal fluence level. In low-dose CT, relatively low I₀ increases the relative signal-to-noise ratio. Here, we focus on CT measurements that are reconstructed into an image estimate A using filter-back projection (FBP). In conventional denoising, the trade-off between noise and resolution is often controlled with a single parameter. For example, a simple low-pass filter with a controllable cut-off frequency can effectively remove the high-frequency noise but with a sacrifice in spatial resolution. We seek to provide a generalized denoising method that has similar control of image properties such that

$$\tilde{μ} ( σ ) = f ( \hat{μ} , σ ) , \qquad ( 1 )$$

where \hat{μ} is the denoised image with hyper-parameter σ that represents a particular level of bias in the restored image. Various bias metrics might be applied; however, we consider the familiar case where σ represents a measure of spatial resolution. Thus, the general denoising problem is then summarized as finding the f(·) that can efficiently decrease the noise while controlling the overall blur by minimizing the following loss function,

$$L ( μ , \hat{μ} , σ ) = {\vert f ( \hat{μ} , σ ) - G ( σ ) * μ \vert}^{2} \qquad ( 2 )$$

where μ is the ground truth image (or, alternately a normal dose, low noise CT scan), and G(σ) is a Gaussian kernel with standard deviation σ. The loss function evaluates the mean squared error (MSE) between the estimated denoised image and the parameterized, blurred ground truth.
1.2. Neural Network Denoising with Controllable Spatial Resolution

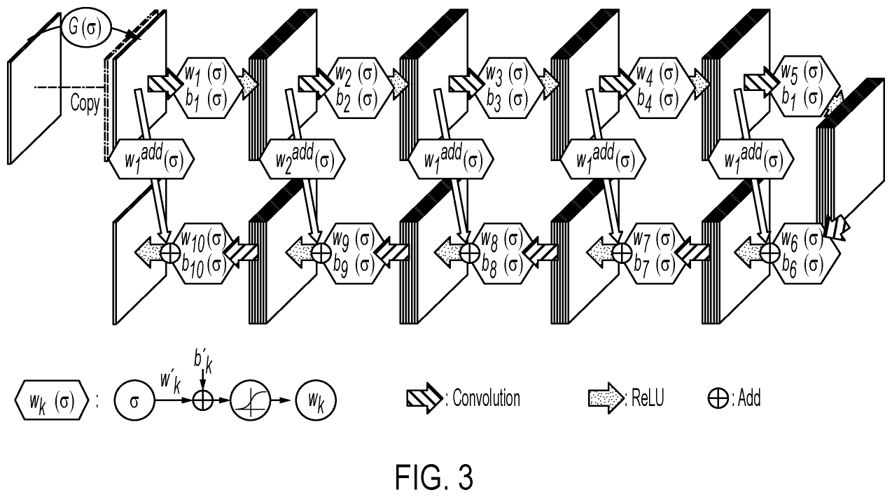

A number of studies have investigated learning-based noise reduction in low-dose CT images, including the residual encoderdecoder convolutional neural network (RED-CNN) and the KAIST-Net. In this example, we adopt the overall architecture of the RED-CNN and add a second input for spatial resolution tuning (FIG. 3).

The RED-CNN incorporates serial 2D convolutional and deconvolutional layers as symmetric encoder and decoder components. Each convolutional/deconvolutional layer is followed by a rectified linear units (ReLU) activation layer:

$${μ}_{k + 1} = ReLU ( {m}_{k} * {μ}_{k} + {a}_{k} ) , ReLU ( u ) = m ( u , 0 ) \qquad ( 3 )$$

where mₖ denotes the convolutional kernels and aₖ denotes the ReLU offsets in the k^{th} convolutional layer. The notations μₖ₊₁,μₖ∈ [symbol] represent the output and input of the k^{th} layer, respectively. The stride of both convolution and deconvolutional layers are fixed to 1 to avoid down-sampling/up-sampling, and the size of each image/feature map in the output is consistent with the original image size. As a result, the implementations of the convolution and deconvolutional layers are essentially the same, and are denoted with convolution operator *. The filter number of all convolutional layers is a constant C except for the last which is 1.

In this example, the desired spatial resolution level u is also an input to the network in addition to the low-dose CT image. Similarly, an additional blurred noisy image is generated with Gaussian filter and stacked with the original image as input to the convolutional layers. To interact with the convolutional layer, the single scalar is expanded through a fully-connected layer with sigmoid activation (Sigmoid (u)=(1+e^{-u})⁻¹) to generate a series of weights and biases wₖ(σ),bₖ(σ) that linearly transform the output of the convolutional layer:

$${w}_{k} ( σ ) = Sigmoid ( {w}_{k}^{′} + {b}_{k}^{′} ) , {b}_{k} ( σ ) = Sigmoid ( {w}_{k}^{″} σ + {b}_{k}^{″} ) , \qquad ( 4 )$$

$${μ}_{k + 1}^{′} = ( {w}_{k} ( σ ) ⊗ 1 ) ⊙ ( {m}_{k} * {μ}_{k} + {a}_{k} ) + {b}_{k} ( σ ) ⊗ 1 ,$$

$${μ}_{k + 1} = ReLU ( {μ}_{k}^{′} + 1 ) \qquad ( 5 )$$

where {wₖ(σ),bₖ(σ)}⊂ [symbol] are channel-wise weights and offsets. Each weight or bias is reshaped to the same size as the image as a Kronecker product of the compact weights and a vector of all ones 1∈ [symbol] . The output of the convolutional layer is weighted and offset before the ReLU activation is applied. Note that all {wₖ(σ),bₖ(σ)} are dependent on the input σ through a generalized nonlinear model, where

$$\{ {w}_{k}^{′} , {b}_{k}^{′} , {w}_{k}^{″} , {b}_{k}^{″} \}$$

are trainable parameters.

In RED-CNN, shortcut connections are introduced to preserve detailed structural information and facilitate deeper network training for residual estimation. In this work, we keep the shortcut connections structures. Assuming the encoding process extracts different features in the hidden layers that may be retained or removed at different spatial resolution levels, we add a weighting to the corresponding decoding layer.

$${μ}_{K - k + 1} = ReLU ( ( {w}_{k}^{add} ⊗ 1 ) ⊙ {μ}_{k} + {μ}_{K - k}^{′} ) \qquad ( 6 )$$

where K denotes the total number of layers. The k^{th} layer output μₖ is weighted and added into the input to the ReLU activation in Layer K-k. The weights

$${w}_{k}^{add}$$

are dependent on the spatial resolution parameter σ following Equation (4). In the last layer, only the Gaussian-blurred noisy image is connected to the add layer because the estimation target is the Gaussian-blurred ground truth. Thus the overall structure of the network seeks to minimize the residual between low-dose and normal-dose images at a certain spatial resolution level.

### 1.3. Neural Network Training

We used 742 2D normal-dose CT images of 512×512 pixels in the training set, and 74 images in the testing set downloaded from the Cancer Imaging Archive (TCIA). Low-dose scans were simulated through reprojection and addition of Poisson noise with incident fluence I₀=1×10⁴. Corresponding low-dose CT image volumes were formed via the FBP reconstructions of these noisy measurements. The proposed model was trained with an augmented training set where 5 different Gaussian blur kernel with standard deviations σ=0,0.5,1,1.5,2 pixels were applied. The overall empirical loss function is written as,

$$ℒ ( \{ {μ}_{n} \} ; {𝒲}_{f ( \cdot )} ) = {\sum }_{j} {\sum }_{n} {\vert f ( {\hat{μ}}_{n} , {σ}_{j} ) - G ( {σ}_{j} ) * {μ}_{n} \vert}^{2} .$$

where [symbol] _{f(·)} includes all the trainable parameters including the convolutional filters mₖ, convolutional layer biases aₖ, and σ-related weights and biases

$$\{ {w}_{k}^{′} , {b}_{k}^{′} , {w}_{k}^{″} , {b}_{k}^{″} , {w}_{k}^{add} \} .$$

We used 300 epochs of the Adam algorithm 8 to minimize the loss function. To retain the possible location-dependent noise features, the entire CT was used (e.g. not image patches). The model was tested with σ between 0 and 2 pixels with 0.25 pixel interval, where half of the testing values were not included in the training set. A RED-CNN model was trained using the same dataset and compared with the proposed model.

### 1.4. Task-Based Performance Evaluation

To investigate the performance of the proposed sCNN across σ values, we adopted a task-based metric. In particular, we focused on a detection task, where a specific signal g is either present or absent on a background image. We adopted a nonprewhitening observer model and used the following generalized form to compute the detectability index d^{t2}=2(ĝ^{τ} ĝ)²/(ĝ^{τ}(A_{bg}+A_{g})ĝ), where ĝ denotes the mean response to the signal g,A_{g} is the covariance matrix of ĝ, and A_{bg} is the covariance matrix of the signal-absent background.

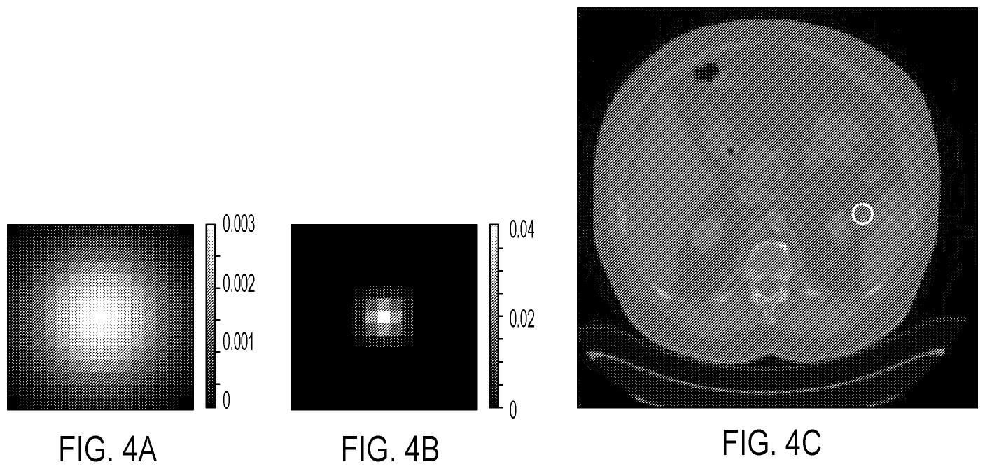

Two different task objects emulating a large low-contrast lesion and a small calcification (FIG. 4) were inserted in the normal dose CT images in the testing set. For each image, 10 low-dose images were simulated with Poisson noise injection in projection data. The σ ranged in [0,4] for the high-contrast task and in [0,8] for the low-contrast task. Detectability indices were computed in the denoised estimates using the proposed sCNN and the RED-CNN. The relative detectability indices were computed by normalization to the corresponding RED-CNN detectability indices.

### 2. Results

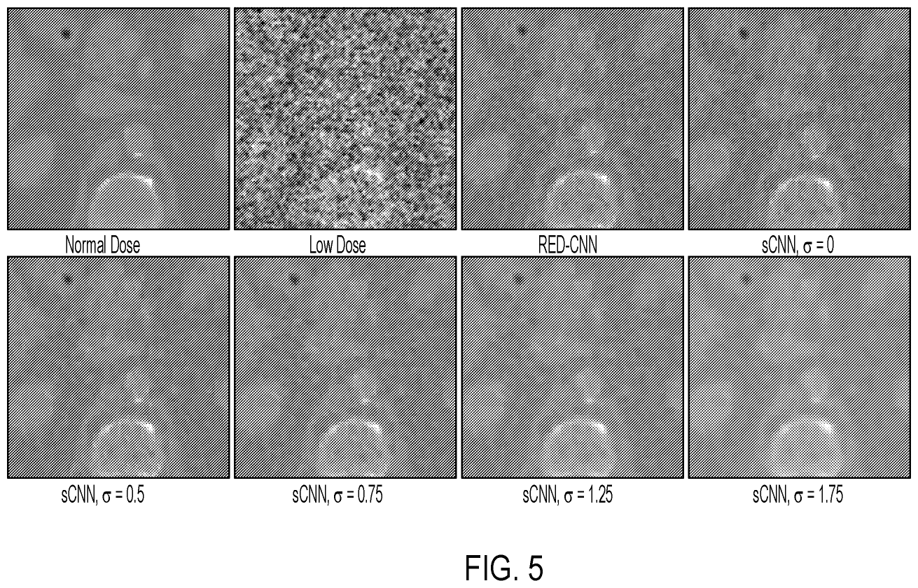

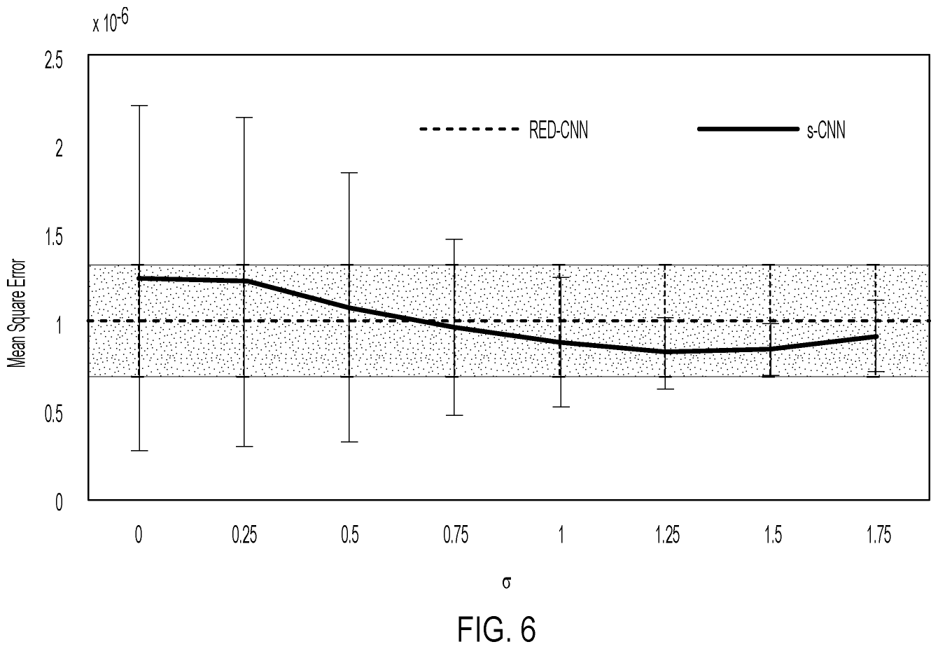

A representative sample of the reference normal-dose image, the simulated low-dose image, and the denoised images using RED-CNN and the proposed sCNN models are shown in FIG. 5. Both the RED-CNN and sCNN results show significantly reduced noise and maintain most of the structures. Compared with the RED-CNN denoised image, the sCNN result with σ=0 shows comparable image quality. Additionally, the sCNN provides further denoising ability with lower “resolution” for increasing σ values. FIG. 6 displays the mean and variance of the measured mean-squared errors (MSE, between denoised and truth) in a central 300-by-300 region-of-interest in 74 testing images. The blue line and bar show the average MSE and the one standard deviation interval of the RED-CNN estimates compared with the ground truth, while the orange ones show those of the sCNN results. We observed that the mean MSE decreases with the increased σ until the turning point σ=1.25. The variance of the measured MSE among testing images decreases with the increased σ. This demonstrates that the sCNN can establish a noise-bias trade-off with the proposed network structure.

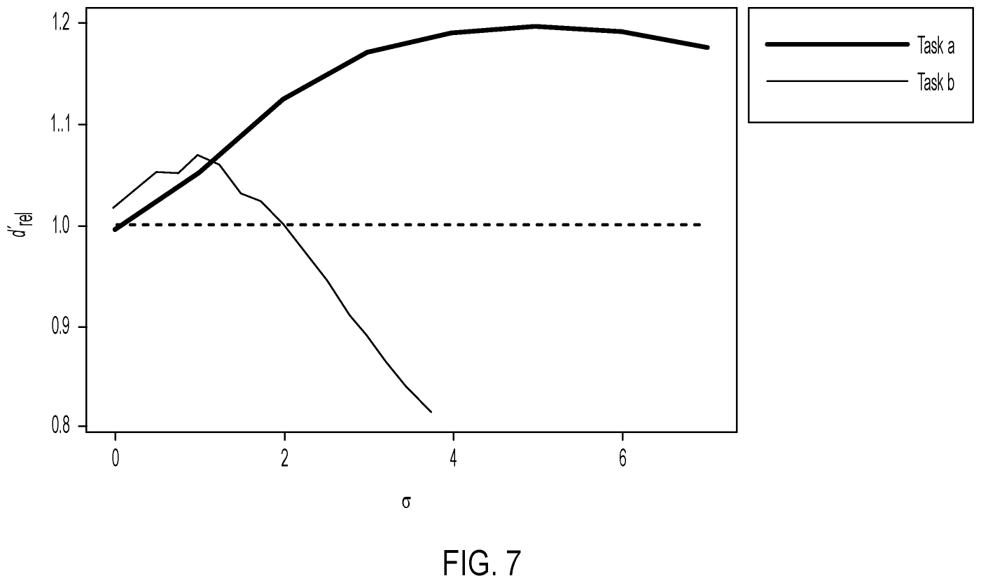

The average relative detectability indices are plotted in FIG. 7. The task-based evaluation shows that with the sCNN model, the detectability is optimized at different σ values for the low- and high-frequency imaging tasks. Moreover, the optimized detectability is greater than the RED-CNN (green dotted line) which has no resolution control. The optimal σ of the low-frequency low-contrast imaging task is higher than the small high-contrast task. This is expected because the low-frequency imaging task is less sensitive to spatial resolution loss and benefits from additional noise reduction.

### 3. Conclusions and Discussion

In this example, we disclose a CNN for noise reduction in low-dose CT with spatial resolution control and evaluated the network performance with general quantitative metrics of MSE and task-based detectability. The results showed that introduction of spatial resolution control provides controllability over how aggressively the noise reduction is applied. This allows for personalized selection of the trade-off between spatial resolution and noise, and permits customization for specific diagnostic tasks. We expect that such controllability also yields an opportunity to investigate and control more general biases associated with machine learning methods. A neural network with this kind of parameterization may permit tuning of the approach to both explore and limit the introduction of false features in a reconstruction. For example, a feature present across u values may be less likely to be false if presenting in both less and more aggressively applied noise reduction. Such questions and analysis are the subjects of ongoing efforts.

Some further aspects are defined in the following clauses:

Clause 1: A computer-implemented method of producing a processed test image having a controlled image quality level. The method comprising: receiving at least one selected input value of at least one control parameter in a trained electronic neural network, wherein the selected input value determines an image quality level of the processed test image, which image quality level comprises relative amounts of at least one noise measure and at least one bias measure in the processed test image; passing test image data through the trained electronic neural network; and, outputting from the trained electronic neural network the processed test image, thereby producing the processed test image having the controlled image quality level.

Clause 2: The computer-implemented method of Clause 1, wherein the control parameter represents a level of bias in the processed test image.

Clause 3: The computer-implemented method of Clause 1 or Clause 2, wherein the control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof.

Clause 4: The computer-implemented method of any one of the preceding Clauses 1-3, wherein the bias measure comprises a spatial resolution level, a blurriness level, or another misrepresentation of an image volume.

Clause 5: The computer-implemented method of any one of the preceding Clauses 1-4, wherein the processed test image comprises a denoised image.

Clause 6: The computer-implemented method of any one of the preceding Clauses 1-5, wherein the trained electronic neural network comprises a loss function having the formula:

$$L ( μ , \hat{μ} , σ ) = {\vert f ( \hat{μ} , σ ) - G ( σ ) * μ \vert}^{2}$$

where μ is a ground truth image, μ is an image estimate using filter-back projection (FBP), σ is the control parameter, and G(σ) is a Gaussian kernel with standard deviation σ.

Clause 7: The computer-implemented method of any one of the preceding Clauses 1-6, wherein the loss function evaluates a mean squared error (MSE) between the image estimate and the parameterized ground truth image.

Clause 8: The computer-implemented method of any one of the preceding Clauses 1-7, wherein the trained electronic neural network comprises a convolutional neural network (CNN).

Clause 9: The computer-implemented method of any one of the preceding Clauses 1-8, wherein the test image data comprises one or more images selected from the group consisting of: a magnetic resonance (MR) image, a computed tomography (CT) image, a single photon emission computed tomography (SPECT) image, a positron emission tomography (PET) image, and a microscopy image.

Clause 10: The computer-implemented method of any one of the preceding Clauses 1-9, wherein the test image data comprises acquired computed tomography (CT) projection data of an object.

Clause 11: The computer-implemented method of any one of the preceding Clauses 1-10, wherein the processed test image comprises a reconstructed CT image.

Clause 12: The computer-implemented method of any one of the preceding Clauses 1-11, wherein the bias measure is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

Clause 13: The computer-implemented method of any one of the preceding Clauses 1-12, comprising receiving x-ray CT data at one or more x-ray detectors configured and positioned to detect x-ray energy transmitted through the object from one or more x-ray energy sources to generate the acquired CT projection data of the object.

Clause 14: The computer-implemented method of any one of the preceding Clauses 1-12, wherein the object comprises a subject.

Clause 15: A system for producing a processed test image having a controlled image quality level using an electronic neural network. The system comprising: a processor; and a memory communicatively coupled to the processor, the memory storing instructions which, when executed on the processor, perform operations comprising: receiving at least one selected input value of at least one control parameter in a trained electronic neural network, wherein the selected input value determines an image quality level of the processed test image, which image quality level comprises relative amounts of at least one noise measure and at least one bias measure in the processed test image; passing test image data through the trained electronic neural network; and, outputting from the trained electronic neural network the processed test image to thereby produce the processed test image having the controlled image quality level.

Clause 16: The system of Clause 15, wherein the control parameter represents a level of bias in the processed test image.

Clause 17: The system of Clause 15 or Clause 16, wherein the control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof.

Clause 18: The system of any one of the preceding Clauses 15-17, wherein the bias measure comprises a spatial resolution level, a blurriness level, or another misrepresentation of an image volume.

Clause 19: The system of any one of the preceding Clauses 15-18, wherein the processed test image comprises a denoised image.

Clause 20: The system of any one of the preceding Clauses 15-19, wherein the trained electronic neural network comprises a loss function having the formula:

$$L ( μ , \hat{μ} , σ ) = {\vert f ( \hat{μ} , σ ) - G ( σ ) * μ \vert}^{2}$$

where μ is a ground truth image, \hat{μ} is an image estimate using filter-back projection (FBP), σ is the control parameter, and G(σ) is a Gaussian kernel with standard deviation σ.

Clause 21: The system of any one of the preceding Clauses 15-20, wherein the loss function evaluates a mean squared error (MSE) between the image estimate and the parameterized ground truth image.

Clause 22: The system of any one of the preceding Clauses 15-21, wherein the trained electronic neural network comprises a convolutional neural network (CNN).

Clause 23: The system of any one of the preceding Clauses 15-22, wherein the test image data comprises one or more images selected from the group consisting of: a magnetic resonance (MR) image, a computed tomography (CT) image, a single photon emission computed tomography (SPECT) image, a positron emission tomography (PET) image, and a microscopy image.

Clause 24: The system of any one of the preceding Clauses 15-23, wherein the test image data comprises acquired computed tomography (CT) projection data of an object.

Clause 25: The system of any one of the preceding Clauses 15-24, wherein the processed test image comprises a reconstructed CT image.

Clause 26: The system of any one of the preceding Clauses 15-25, wherein the bias measure is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

Clause 27: The system of any one of the preceding Clauses 15-26, wherein the non-transitory computer-executable instructions which, when executed by the electronic processor, further perform at least: receiving x-ray CT data at one or more x-ray detectors configured and positioned to detect x-ray energy transmitted through the object from one or more x-ray energy sources to generate the acquired CT projection data of the object.

Clause 28: The system of any one of the preceding Clauses 15-27, wherein the object comprises a subject.

Clause 29: A computer readable media comprising non-transitory computer executable instruction which, when executed by at least electronic processor, perform at least: receiving at least one selected input value of at least one control parameter in a trained electronic neural network, wherein the selected input value determines an image quality level of the processed test image, which image quality level comprises relative amounts of at least one noise measure and at least one bias measure in the processed test image; passing test image data through the trained electronic neural network; and, outputting from the trained electronic neural network the processed test image to thereby produce the processed test image having the controlled image quality level.

Clause 30: The computer readable media of Clause 29, wherein the control parameter represents a level of bias in the processed test image.

Clause 31: The computer readable media of Clause 29 or Clause 30, wherein the control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof.

Clause 32: The computer readable media of any one of the preceding Clauses 29-31, wherein the bias measure comprises a spatial resolution level, a blurriness level, or another misrepresentation of an image volume.

Clause 33: The computer readable media of any one of the preceding Clauses 29-32, wherein the processed test image comprises a denoised image.

Clause 34: The computer readable media of any one of the preceding Clauses 29-33, wherein the trained electronic neural network comprises a loss function having the formula:

$$L ( μ , \hat{μ} , σ ) = {\vert f ( \hat{μ} , σ ) - G ( σ ) * μ \vert}^{2}$$

where μ is a ground truth image, \hat{μ} is an image estimate using filter-back projection (FBP), σ is the control parameter, and G(σ) is a Gaussian kernel with standard deviation σ.

Clause 35: The computer readable media of any one of the preceding Clauses 29-34, wherein the loss function evaluates a mean squared error (MSE) between the image estimate and the parameterized ground truth image.

Clause 36: The computer readable media of any one of the preceding Clauses 29-35, wherein the trained electronic neural network comprises a convolutional neural network (CNN).

Clause 37: The computer readable media of any one of the preceding Clauses 29-36, wherein the test image data comprises one or more images selected from the group consisting of: a magnetic resonance (MR) image, a computed tomography (CT) image, a single photon emission computed tomography (SPECT) image, a positron emission tomography (PET) image, and a microscopy image.

Clause 38: The computer readable media of any one of the preceding Clauses 29-37, wherein the test image data comprises acquired computed tomography (CT) projection data of an object.

Clause 39: The computer readable media of any one of the preceding Clauses 29-38, wherein the processed test image comprises a reconstructed CT image.

Clause 40: The computer readable media of any one of the preceding Clauses 29-39, wherein the bias measure is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

Clause 41: The computer readable media of any one of the preceding Clauses 29-40, wherein the non-transitory computer-executable instructions which, when executed by the electronic processor, further perform at least: receiving x-ray CT data at one or more x-ray detectors configured and positioned to detect x-ray energy transmitted through the object from one or more x-ray energy sources to generate the acquired CT projection data of the object.

Clause 42: The computer readable media of any one of the preceding Clauses 29-41, wherein the object comprises a subject.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, cranial implant devices, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A computer-implemented method of producing a processed test image having a controlled image quality level, the method comprising:
receiving at least one selected input value of at least one control parameter in a trained electronic neural network, wherein the selected input value determines an image quality level of the processed test image, which image quality level comprises relative amounts of at least one noise measure and at least one bias measure in the processed test image;
passing test image data through the trained electronic neural network; and,
outputting from the trained electronic neural network the processed test image, thereby producing the processed test image having the controlled image quality level.

2. The method of claim 1, wherein the control parameter represents a level of bias in the processed test image.

3. The method of claim 1, wherein the control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof.

4. The method of claim 1, wherein the bias measure comprises a spatial resolution level, a blurriness level, or another misrepresentation of an image volume.

5. The method of claim 1, wherein the processed test image comprises a denoised image.

6. The method of claim 1, wherein the trained electronic neural network comprises a loss function having the formula:
$$L ( μ , \hat{μ} , σ ) = {\vert f ( \hat{μ} , σ ) - G ( σ ) * μ \vert}^{2}$$
where μ is a ground truth image, \hat{μ} is an image estimate using filter-back projection (FBP), σ is the control parameter, and G(σ) is a Gaussian kernel with standard deviation σ.

7. The method of claim 6, wherein the loss function evaluates a mean squared error (MSE) between the image estimate and the parameterized ground truth image.

8. The method of claim 1, wherein the trained electronic neural network comprises a convolutional neural network (CNN).

9. The method of claim 1, wherein the test image data comprises one or more images selected from the group consisting of: a magnetic resonance (MR) image, a computed tomography (CT) image, a single photon emission computed tomography (SPECT) image, a positron emission tomography (PET) image, and a microscopy image.

10. The method of claim 1, wherein the test image data comprises acquired computed tomography (CT) projection data of an object.

11. The method of claim 1, wherein the processed test image comprises a reconstructed CT image.

12. The method of claim 1, wherein the bias measure is selected from the group consisting of: a drift in x-ray energy source, a drift in an x-ray energy detector, an incomplete scatter rejection, an inexact scatter correction, an inexact x-ray energy source calibration, a filtration of x-rays from an x-ray energy source, a physical effect induced by the object, a reconstruction algorithm effect, a beam hardening effect, a scattering effect, and a detector effect.

13. The method of claim 1, comprising receiving x-ray CT data at one or more x-ray detectors configured and positioned to detect x-ray energy transmitted through the object from one or more x-ray energy sources to generate the acquired CT projection data of the object.

14. The method of claim 1, wherein the object comprises a subject.

15. A system for producing a processed test image having a controlled image quality level using an electronic neural network, the system comprising:
a processor; and
a memory communicatively coupled to the processor, the memory storing instructions which, when executed on the processor, perform operations comprising:
receiving at least one selected input value of at least one control parameter in a trained electronic neural network, wherein the selected input value determines an image quality level of the processed test image, which image quality level comprises relative amounts of at least one noise measure and at least one bias measure in the processed test image;
passing test image data through the trained electronic neural network; and,
outputting from the trained electronic neural network the processed test image to thereby produce the processed test image having the controlled image quality level.

16. The system of claim 15, wherein the control parameter represents a level of bias in the processed test image; or wherein the control parameter represents a property selected from the group consisting of: a resolution level, a noise level, a deconvolution level, a task-specific performance measure, a detectability measure, a variance level, a perceptual loss measure, a similarity index measure, a combination thereof, and a ratio thereof.

17. (canceled)

18. The system of claim 15, wherein the bias measure comprises a spatial resolution level, a blurriness level, or another misrepresentation of an image volume.

19. (canceled)

20. The system of claim 15, wherein the trained electronic neural network comprises a loss function having the formula:
$$L ( μ , \hat{μ} , σ ) = {\vert f ( \hat{μ} , σ ) - G ( σ ) * μ \vert}^{2}$$
where μ is a ground truth image, \hat{μ} an image estimate using filter-back projection (FBP), σ is the control parameter, and G(σ) is a Gaussian kernel with standard deviation σ.

21. The system of claim 20, wherein the loss function evaluates a mean squared error (MSE) between the image estimate and the parameterized ground truth image.

22.-28. (canceled)

29. A computer readable media comprising non-transitory computer executable instruction which, when executed by at least electronic processor, perform at least:
receiving at least one selected input value of at least one control parameter in a trained electronic neural network, wherein the selected input value determines an image quality level of the processed test image, which image quality level comprises relative amounts of at least one noise measure and at least one bias measure in the processed test image;
passing test image data through the trained electronic neural network; and,
outputting from the trained electronic neural network the processed test image to thereby produce the processed test image having the controlled image quality level.

30.-42. (canceled)
